(19) United States
(12) Patent Application Publication
(10) Pub. No.: US 2008/0207932 A1
(43) Pub. Date: Aug. 28, 2008

(54) Kiwifruit Oil Extraction Method And Product

(75) Inventors: Bruce William Donaldson, Auckland (NZ); Karl-Werner Quirin, Rehlingen (DE)

Correspondence Address: BROOKS KUSHMAN P.C., 1000 TOWN CENTER, TWENTY-SECOND FLOOR, SOUTHFIELD, MI, 48075, US

(73) Assignee: VITAL FOOD PROCESSORS LIMITED, Auckland (NZ)

(21) Appl. No.: 11/912,724

(22) PCT Filed: May 1, 2006
(86) PCT No.: PCT/NZ2006/000092

§ 371 Date: Jan. 8, 2008

(30) Foreign Application Priority Data
Apr. 29, 2005 (NZ) 539698

(51) Int. Cl. C11B 1/10; A01H 5/10

(52) U.S. Cl. 554/9; 800/298

(57) ABSTRACT

A process of producing kiwifruit seed oil involves first sterilizing the picked and selected ripe kiwifruit by flame, chlorine wash, or ozone impregnated water wash, followed by a fresh water rinse. The fruit is then cut using a water laser to minimize seed damage. Next the skin is separated from the fruit. Then the pulp is gently separated from the seed ensuring minimal seed coat damage. The resulting seed is cleaned whilst ensuring minimal seed coat damage. Then seed is then dried using cool temperature drying. The seed is stored at a cool temperature until it is needed for oil extraction. Crushing of the seed takes place immediately prior to the super critical extraction process. Super critical extraction using liquid carbon dioxide extracts the kiwifruit oil from the crushed seeds. The oil is the stabilized using a natural antioxidant such as rosemary oil. The stabilized oil can then be (a) encapsulated into soft cap gelatin capsules or (b) emulsified and then dried onto a carrier base using a cold temperature drying process or (c) used in the production of a food product.

### FIELD OF INVENTION

The present invention relates to a method for the extraction and purification of kiwifruit seed oil and a product manufactured using this method.

### BACKGROUND OF INVENTION

Alpha-linolenic acid (ALA) is an essential fatty acid. It is the precursor of longer chain fatty acids such as eicosapentaenoic acid (EPA) and docosahexaenoic acid (DHA). It has recently become recognised that ALA is metabolised by pathways which may be quantitatively more important than the formation of DHA. For example, ALA is extensively β-oxidised in mammals including humans, especially on diets rich in polyunsaturated fatty acids.

ALA appears to play an important role in the nervous system (carbon recycling) for the synthesis of saturated and monounsaturated fatty acids and cholesterol, with estimates of the proportion of ALA being metabolised via this pathway being several fold greater than that to DHA.

Over the past 20 years there has been an increasing interest in the fatty acids derived from ALA, the long chain omega-3 fatty acids, which are important for optimal function of a variety of tissues.

ALA is an essential fatty acid because mammals do not possess the ability to insert double bonds in 18-carbon polyunsaturated fatty acids (PUFA) between the methyl end and the middle of the molecule. ALA has three cis double bonds in positions 9-10, 12-13, 15-16 counting from the carboxyl end of the fatty acid (all cis, 9,12,15-octadecatrienoic acid). The position of the first double bond from the methyl end of the molecule has lead to describing ALA as an omega-3 (or n-3) poly-unsaturated fatty acid (PUFA), since the first double bond is 3 carbons from the methyl (omega) end of the fatty acid. An alternative nomenclature is to describe ALA as 18:3 omega-3 or 18:3 n-3 (carbon atoms: double bonds, omega-3 or n-3 representing the position of the first double bond counting from the methyl end).

Linoleic acid (Omega 6) is important for growth, reproduction, and skin function and as the precursor of arachidonic acid, which is the main precursor of the eicosanoids, such as thromboxane, prostaglandins, prostacyclins and leukotrienes.

ALA (Omega 3), through its metabolite DHA, is essential for normal visual function and brain function via effects on membrane fluidity (membrane order) which can influence the function of membrane receptors such as rhodopsin regulation of membrane-bound enzymes (Na/K-dependent ATPase) and in signal transduction via effects on inositol phosphates, DAG and protein kinase C. Both essential fatty acids are important in membrane structure and function since their long chain PUFA derivatives are principally located in cell membranes phospholipids. There has been considerable discussion whether the true essentiality resides with the essential fatty acids or with their long chain derivatives (arachidonic acid in the case of the omega 6 family and EPA and DHA in the case of the omega-3 family). Specific functions have been described for linoleic acid, since there is evidence that it is required for optimal skin function.

Kiwifruit contains approximately 3% of the weight of the mature fruit as seed. This seed yields approx 35% of its weight as oil when processed. Kiwifruit seed oil is particularly rich in Alpha Linolenic Acid and Linoleic Acid, being present as approximately 70% and 15% weight/weight respectively.

Other manufacturers have used harsher methods of isolating the seeds from kiwifruit, which invariably results in contamination and spoilage of the seeds. Such methods include traditional fruit pulping methods, which generally comprise hammer milling the fruit and then separating the skin and pulp from the seed using screens and water. However, this extraction processing technology is unsympathetic to the physical condition of the seed and results in a significant proportion of the seed being damaged in the process. The damage is essentially in the form of impact damage, which tends to crack or in extreme cases break the seed coat.

Also, omega 3 is highly susceptible to oxidative damage, which can be measured by the peroxide levels in the oil. Damage to the seed coat facilitates oxidation degradation of the oil content of the seed during storage prior to oil extraction, resulting in high peroxide values post extraction. Damage to the seed coat is also thought to expose the oil to the enzymes in the kiwifruit pulp, which when given access to the seed contents, can quickly lead to the deterioration of the quality of the oils, even before they are extracted. Enzymes remaining in the isolated oil can detrimentally affect the shelf life of the resultant product.

Oxidative damage can be contained by stabilizing the oil with antioxidants after extraction, however synthetic additives are commonly used as stabilizing agents in fruit seed oil products. There is a need to produce a natural product without the use of synthetic preserving or antioxidant agents, which can be consumed on a regular basis without the risk of side effects.

### OBJECT OF INVENTION

It is therefore an object of the present invention to address the foregoing problems and to provide a method for the extraction and purification of kiwifruit seed oil and a product manufactured using this method, or at least to provide the public with a useful choice.

### STATEMENT OF INVENTION

According to one aspect of the present invention there is provided a method for extracting oil from kiwifruit seeds including the following steps:

a) Sterilizing the external surface of the fruit;

b) Separating the skin from the pulp and seed without damaging the seed coat;

c) Separating the seed from the pulp by soft mechanical screening;

d) Cold drying or freeze drying the isolated seed;

e) Crushing the seed immediately prior to oil extraction;

f) Using supercritical CO₂-extraction to extract the oil from the seed;

g) Stabilization of the oil during or after extraction using a natural antioxidant.

In a preferred embodiment the seed oil may be extracted from the seed of Actinidia deliciosa or Actinidia cheninensis.

Preferably, the kiwifruit is sterilized by the use of a thermal process, followed by a chlorine wash and/or an ozone impregnated water wash and a fresh water rinse.

Preferably, the fruit is cut in half prior to the separation of the skin from the pulp, using a water laser or similar in order to prevent damage to the seed.

Preferably, the seed and fruit pulp may be separated from the skin by way of a twin converging belt press.

Preferably, the seed is separated from the pulp by way of a soft screening process.

Preferably, after the seed is separated from the pulp by screening, the separated seed is cleaned using pectinase, screens and water. In a preferred embodiment the seed may be soaked for up to 12 hours in a pectinase solution to separate any remaining pulp from the seed and then rinsed with water on a screen.

In a preferred embodiment the seed may be dried by freeze drying to prevent heat damage and oxidation to the oil content of the seed, prior to crushing the seed. Alternatively, the seed may also be dried by other methods known in the art, for example, conventional air drying, modified atmosphere drying, or fluidised bed drying. After drying the seeds may be stored under cool temperatures or frozen until required for crushing immediately prior to the oil extraction process.

Preferably, the temperature of the fruit, seed and oil is maintained below 30° C. throughout the entire oil extraction process.

According to a further aspect of the present invention there is provided a kiwifruit seed oil manufactured according to the method of the present invention.

In a preferred embodiment the oil may be blended with one or more natural antioxidants.

In a preferred embodiment the antioxidant blended with the seed oil may be natural rosemary oil.

Alternatively, the antioxidant blended with the seed oil may be natural Vitamin E.

In a preferred embodiment the oil may be packaged under an oxygen-free atmosphere in order to minimize the occurrence of oxidation.

In a preferred embodiment exposure of the oil to light and temperatures above 4° C. during extraction and packaging of the oil may be avoided.

According to a further aspect of the invention there is provided a method of producing a kiwifruit seed oil which is encapsulated in a soft capsule made of non-gelatinous material.

According to a further aspect of the invention there is provided a method of producing a kiwifruit seed oil wherein the seed oil prepared according to the method of the present invention is further stabilised by drying onto an inert carrier.

In a preferred embodiment the inert carrier may be malto-dextrin, cellulose, and/or cyclo dextrin.

In a preferred embodiment the oil may be dried onto the inert carrier by blending water with the oil and creating an emulsion, the emulsion is then mixed with the inert carrier and the resulting blend dried by methods known in the art.

In a preferred embodiment the blend may be dried by freeze drying or spray drying.

Preferably, the blend is dried by freeze drying.

In a preferred embodiment the blend may be dried and then milled to form a granular powder.

According to a further aspect of the present invention there is provided a food product incorporating the kiwifruit seed oil, or dried blend.

In a preferred embodiment the food product may be an infant formula, breakfast cereal, milkshake mix or the like.

According to a further aspect of the present invention there is provided the use of a kiwifruit seed oil or dried blend manufactured according to the present invention in the manufacture of a medicament for the treatment of a condition resulting from a deficiency of Alpha Linolenic Acid or Linoleic Acid.

In a preferred embodiment the condition resulting from a deficiency of Alpha Linolenic Acid or Linoleic Acid is selected from the group consisting of eczema, psoriasis, dermatitis, bipolar disorder, regional degradation and impairment to brain development in infants.

Further aspects and advantages of the present invention will become apparent from the ensuing description which is given by way of example only.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning. That is, it will be taken to mean an inclusion of not only the listed components it directly references, but also other non-specified components or elements. This rationale will also be used when the term ‘comprised’ or ‘comprising’ is used in relation to one or more steps in a method or process.

### BRIEF DESCRIPTION OF DRAWINGS

Further aspects of the present invention will become apparent from the following description which is given by way of example only and with reference to the accompanying drawing in which:

FIG. 1 is a flow diagram depicting a method of kiwifruit seed oil extraction.

### DETAILED DESCRIPTION

The invention relates to the production of a stable kiwifruit seed oil by the combination of a gentle processing technique to carefully separate the seeds from the skin and pulp of the fruit, and a supercritical CO₂-extraction method to produce a high quality natural omega 3 oil with low peroxide values (caused by oxidative damage) and which is manufactured without the use of synthetic preserving or antioxidant agents.

The kiwifruit which can be used in this invention is any species of the genus Actinidia. However, preferably the green kiwifruit, called Actinidia deliciosa is used (this was formerly called Actinidia cheninensis, however this name has now been given to the yellow kiwifruit).

As a general outline of the method of processing the kiwifruit to isolate the kiwifruit seeds prior to oil extraction, the kiwifruit is processed using a very gentle processing technique which is designed to minimise the damage to the seeds during processing. The kiwifruit may first undergo a pre-treatment process, followed by a dehairing process to remove the hairs from the kiwifruit, a cleaning process to clean the kiwifruit, an alignment process for selectively aligning the kiwifruit for cutting, a cutting process for cutting the kiwifruit into segments (preferably into halves), a pressing process for separating the skin from the pulp and seed, and a separating process to separate the seeds from the pulp of the fruit. Once the seeds have been separated they are cleaned and dried (preferably using freeze drying) and depending on when they are to be used for oil extraction, they may be stored (preferably under cold temperatures or frozen). It is important that the seeds are not crushed until immediately prior to oil extraction, as this minimises any damage which could be done by heat and/or oxidation. After crushing the seed, the oil is extracted using supercritical carbon dioxide extraction. The oil is then stabilised during or after extraction using a natural antioxidant.

In light of the above general description of the apparatus and method, the detailed description will now describe in greater detail both the apparatus and method of a preferred embodiment formed in accordance with the present invention.

A pre-treatment process may be performed if desired which may include the well known steps of ripening, inspecting, grading, and/or sorting of the kiwifruit. With regard to ripening, ripe or mature kiwifruit are preferred and a sugar level of 12±4° Brix is ideal and indicative of ripeness, if the juice of the kiwifruit is desired to be harvested along with the seeds of the kiwifruit. Kiwifruit exceeding this Brix level may be acceptable but are likely to be overly mature or fermenting. Kiwifruit with a Brix level below the ideal, may be artificially ripened before use. Time left in storage may be sufficient—kiwifruit picked at 5° Brix rises to 10.5° Brix in 4-6 weeks in cool storage at 0° C. This fruit will ripen to reach 12° C. or higher upon removal from cool storage. Other changes in chemistry also occur as the kiwifruit ripens so that mature kiwifruit within the ideal range often provide a superior product.

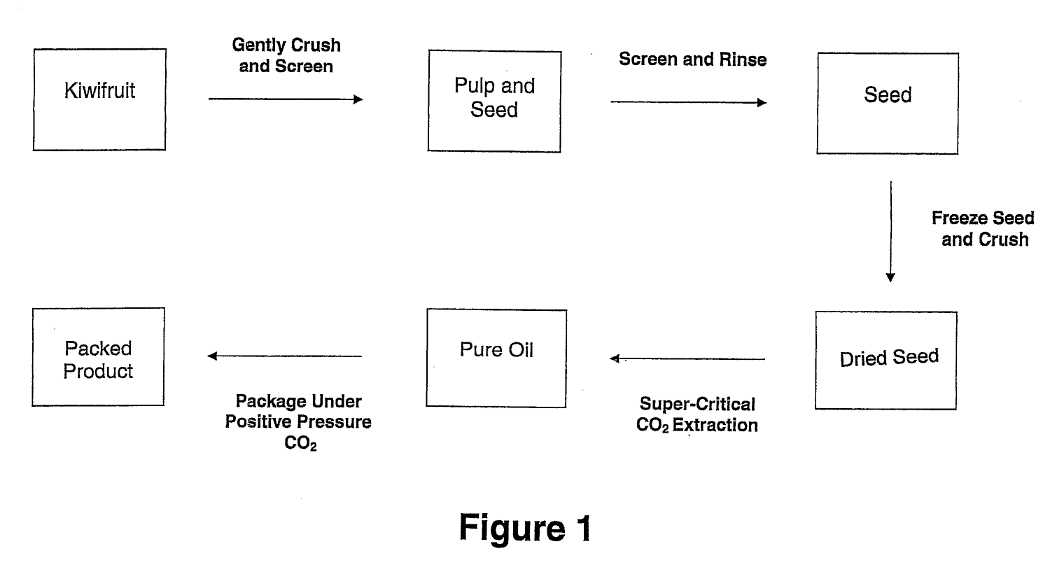

Referring to FIG. 1, upon completion of the pre-treatment process, if conducted, the external surface of the kiwifruit is then sterilized. This preferably involves firstly dehairing the kiwifruit, using a thermal process to remove the hairs. More specifically, the thermal dehairing process may include an oven where a pyrolytic flaming step may be used to singe the hair of the kiwifruit. Preferably, during the flame process, the temperature of the kiwifruit does not become elevated during the burning of the hair. Short burn times and pre-cooling of the kiwifruit can result in the flesh of the kiwifruit, other than that immediately adjacent the skin, not rising above 30° C.

Once the hair of the kiwifruit is removed by the thermal dehairing process, the dehaired kiwifruit are then further sterilized. The fruit may be passed through an assembly having one or more roller brushes for removing any adhering foreign matter including singed hairs from the kiwifruit. Conventional washing techniques may then be employed, one example being the use of a series of spray nozzles. Wash additives aiding cleansing or reducing the bacteria count on the kiwifruit may be employed according to local regulations and requirements. For example, the fruit may be washed by a chlorine wash and/or an ozone impregnated water wash followed by a fresh water rinse.

The sterilized kiwifruit are then conveyed into a hopper, which is generally tapered to form a funnel directing the kiwifruit individually, that is, one by one, into a further conveyor system which conveys the fruit to a cutting assembly. The cutting assembly includes a cutting device such as a water laser or similar in order to prevent damage to the seed. However, it should be apparent to those skilled in the art that other cutting devices are suitable for use with the present invention, including rotating circular blades, reciprocating blades, fluid jet cutting devices, swing blades, etc.

Preferably, the cutting device cuts the kiwifruit substantially in half. Further, the cutting device preferably cuts the kiwifruit along its length as this has been found to result in reduced seed damage. Although the cutting device is described as preferably cutting the kiwifruit in halves lengthwise, it should be noted that the kiwifruit may be cut into multiple sections or sections of other sizes and shapes without departing from the spirit and scope of the present invention. Alternately, the cutting device may be replaced with a soft crushing device able to break the skin of the kiwifruit without causing significant seed or cellular damage to the kiwifruit. For instance, the kiwifruit may be directed between rollers to result in the breakage of the skin of the kiwifruit. For instance, the kiwifruit may be burst by passing the kiwifruit through spaced rollers biased towards each other. This squashes the fruit so the skin is split, the burst kiwifruit substantially intact but readily separable into large fragments. Other bursting methods may be employed.

After the kiwifruit are cut the semi-spherical shaped kiwifruit segments are then passed through a pressing assembly designed to separate the skin from the pulp and seed without damaging the seed coat. Generally described, the pressing assembly is adapted to perform “soft-pulping” operations. The term “soft-pulping” relates to a pulping or comminution process which is relatively mild and gentle compared to many conventional fruit pulping techniques. Soft-pulping is characterised by only a minor proportion (generally less than 5-10%) of seeds being fragmented. Further, there is no significant disintegration or lysis of fruit cells or components. Excluded from the meaning of soft-pulping processes are chemical and/or enzyme lysis methods, thermal techniques, techniques directed to the breaking down of cells, and mechanical techniques which involve excessive pulverisation of fruit material.

In a preferred embodiment, the pressing assembly performs the “soft-pulping” of the kiwifruit by pressing the kiwifruit segments between a twin converging belt press. The press belts may be endless loops rotated about a series of pulleys. The distance separating the press belts preferably decreases in the direction of travel of the kiwifruit. This results in increasing pressing forces being exerted upon the kiwifruit as the kiwifruit travel along the length of the pressing assembly. This action results in the soft pulping of the kiwifruit without significant damage to the seeds.

The pulp generated from the pressing assembly is then directed to a screening process, in order to separate the seeds from the pulp. Generally, the pulp is separated from the seed using a soft mechanical screening technique. This preferably involves the use of a pulp finisher including a rotating flexible impeller which is rotated within a cone shaped screen having apertures of a predetermined size. The size of the apertures is preferably selected to permit the pulp and juice of the kiwifruit to pass through the screen while retaining a substantial portion, if not all, of the seeds within the interior cavity defined by the screen.

Preferably, after the seed is separated from the pulp by screening, the separated seed is then cleaned using pectinase, screens and water. In a preferred embodiment the seed may be soaked for up to 12 hours in a pectinase solution to separate any remaining pulp from the seed and then rinsed with water on a screen.

The isolated seeds are then dried using cool temperatures, for example, by freeze drying.

The use of cold temperatures for drying the seeds is advantageous as it prevents heat and oxidation damage to the oil content of the seeds. Alternatively, the seed may also be dried by other methods known in the art, for example, conventional air drying, modified atmosphere drying, or fluidised bed drying. After drying, if the seeds are not to be used immediately for oil extraction, then they are preferably stored under cool temperatures or frozen until required for oil extraction. Again, this storage of the seeds at cool temperatures prevents heat and oxidation damage to the oil content of the seeds.

When the oil extraction process is ready to be carried out, the seeds are crushed immediately prior to the oil extraction process. It is important that the seeds are not crushed until they are immediately required, as this prevents head and oxidation damage to the oil content of the seeds. Oil extraction is then carried out by using methods known to those skilled in the art. Preferably however, the oil extraction process used is super critical carbon dioxide extraction, using liquid carbon dioxide.

An analysis of oil extracted from kiwifruit seeds in the manner described by this invention was carried out. The results of the analysis are listed in the table below:

| | | | |
|---|---|---|---|
| Feature | Method | Limits | Value |

Fatty acid composition:

| | | | |
|---|---|---|---|
| Palmitic Acid | GC FID, | n.s. | 5.0% |
| C 16:0 | 2 21.012.03 | | |
| Palmitoleic Acid | | n.s. | n.d. % |
| C 16:1 w7 | | | |
| Stearic Acid | | n.s. | 2.5% |
| C18:0 | | | |
| Oleic Acid | | n.s. | 11.6% |
| C18:1 w9 | | | |
| Vaccenic Acid | | n.s. | 0.29% |
| C18:1 w7 | | | |
| Linoleic Acid | | 12-17 | 14.9% |
| C18:2 w6 | | | |
| Alpha Linolenic Acid | | >=65 | 65.3% |
| C18:3 w3 | | | |

Fat numbers:

| | | | |
|---|---|---|---|
| Unsaponifiables | 21.020.01 | n.s. | 1.3% |
| Peroxide value | 21.015.01, | n.s. | 2.7 meq/kg |
| | Wheeler | | |
| Acid value | 21.016.01 | n.s. | 3.8 |
| Saponification number | 21.017.01 | n.s. | 196 |
| Ester number | = VZ − SZ | n.s. | 192 |
| Refractive index (22° C.) | Abbe-Refraktometer | n.s. | 1.4830 |
| Density (20° C.) | Pyknometer | n.s. | 0.9308 g/cm³ |

n.s. = not specified
n.d. = not detected

During or after the oil extraction process, the oil is then blended with a natural antioxidant in order to stabilize the oil. It has been found that a very effective natural antioxidant for stabilizing the oil is natural rosemary oil. Vitamin E may also be used, however natural rosemary oil has been found to be more effective at stabilizing the oil. A stability study was carried out to determine the stability of oil extracted from seeds using conventional processing technology compared to the stability of oil extracted from seeds using the processing technology as described in the present invention, that is, the gentle or soft-pulping technology.

The stability tests showed strong deviations depending on the methods used. For example, seeds which were separated from the kiwifruit pulp and skin by conventional methods, and then cold pressed to produce an oil, reached a peroxide value of greater than 10 in less than or equal to two days. On the other hand, seeds which were separated from the kiwifruit pulp and skin using the method of the present invention, and then cold pressed to produce an oil, reached a peroxide value of greater than 10 in less than or equal to 14 days. The test was then repeated using super critical carbon dioxide oil extraction instead of cold pressing. It was found that seeds which were separated from the kiwifruit pulp and skin by conventional methods, and then subjected to super critical carbon dioxide oil extraction, reached a peroxide value of greater than 10 in less than three months. On the other hand, seeds which were separated from the kiwifruit pulp and skin using the method of the present invention, and then subjected to super critical carbon dioxide oil extraction, reached a peroxide value of greater than 10 in six to nine months. This latter procedure was then repeated, however natural rosemary oil was added to the oil during the super critical carbon dioxide oil extraction process. This resulted in an oil which had a peroxide value of less than 10 for at least 24 months.

This study clearly shows that surprisingly it has been found that using a gentle soft pulping technique to isolate the seeds of the kiwifruit, in combination with super critical carbon dioxide oil extraction, and with the addition of rosemary as an antioxidant during the oil extraction process, an extremely stable kiwifruit seed oil can be produced.

In preferred embodiments of the invention, the kiwifruit, the resulting isolated seeds, and the extracted oil should be maintained at a temperature of less than 30° C. throughout the entire oil extraction process. Preferably however, exposure of the oil to light and temperature above 4° C. should be avoided. In addition, the resulting oil should be packaged under an oxygen free atmosphere in order to minimize the occurrence of oxidation.

Once the oil has been extracted from the kiwifruit, it may be packaged into soft capsules made from non-gelatinous material. These capsules may be used as a dietary supplement or the like, and can be manufactured as necessary to suit particular dietary requirements.

Alternatively, the oil may be further stabilized by drying the oil onto an inert carrier. In a preferred embodiment the inert carrier may be malto-dextrin, cellulose, and/or cyclo dextrin.

In a preferred embodiment the oil may be dried onto the inert carrier by blending water with the oil and creating an emulsion, the emulsion is then mixed with the inert carrier and the resulting blend dried by methods known in the art, such as freeze drying or spray drying. Preferably, the blend is dried by freeze drying. The dried blend may then be milled to form a granular powder.

In a further embodiment of the invention, the extracted kiwifruit seed oil or the dried blend as described above, may be incorporated into a food product. Examples of such products include infant formulas, breakfast cereals, milkshake or other drink mixes or the like.

In a further embodiment of the invention, it is recognized that the extracted kiwifruit seed oil or dried blend as described above could be used in the manufacture of a medicament for the treatment of a condition resulting from a deficiency of Alpha Linolenic Acid or Linoleic Acid. Examples of such conditions include eczema, psoriasis, dermatitis, bipolar disorder, regional degradation and impairment to brain development in infants.

### EXAMPLE 1

A kiwifruit seed oil was extracted from the fruit of Actinidia deliciosa using the following method

1. Fruit was ripened to maturity (all rots or disease being removed)

2. Fruit was sterilized by flame, chlorine wash, ozone impregnated water wash and fresh water rinse

3. Fruit was singulated, then cut using a water laser to minimize seed damage

5. The skin was separated from the fruit

6. Pulp was separated from the seed ensuring minimal seed coat damage

7. Seed was cleaned ensuring minimal seed coat damage

8. Seed was dried using cool temperature drying

9. Dried seed was stored under cool temperature

10. Immediately prior to super critical oil extraction process, the seed was crushed

11. Oil was extracted from the seed using supercritical carbon dioxide extraction

12. Oil was stabilized using rosemary oil as a natural antioxidant

13. The stabilized oil was then encapsulated into soft capsules.

### EXAMPLE 2

A kiwifruit seed oil was extracted from the fruit of Actinidia deliciosa using the method as described in steps 1 to 12 of Example 1. However, instead of step 13, the stabilized oil was emulsified with water then dried under cold temperatures onto a inert carrier base.

### Advantages

The technology of the present invention represents a milestone in the development of fruit seed oil extracts. By the use of low temperatures throughout the processing of the kiwifruit to produce the oil, the sensitive constituents of the fruit are preserved and artifact formation by oxidation is avoided. In addition, the resulting oil extracts from this process are completely natural, and conform to rigorous solvent testing requirements for a number of food ingredients. An additional benefit is that the products meet kosher criteria. A further significant advantage of the kiwifruit seed oil extraction is that it is sterile and needs no preservation due to the absence of water, proteins and sugars, making the resulting products free from bacteria and easy to standardise.

The products resulting from this invention provide a vegetarian alternative to fish oils, and are different from other plant sources of omega 3 as they contain more than 65% omega 3 fatty acids in the form of alpha-linolenic acid (ALA). They are also an attractive alternative to the use of fish oils, which are unpopular due to concerns related to their effect on the sensory profile of the final food product.

Also, the resulting products are completely natural as they do not use any synthetic preserving or antioxidant agents. Therefore, the products can be consumed on a regular, even daily basis without the risk of side effects.

### Variations

Where, in the foregoing description reference has been made to integers or components having known equivalence, then such equivalents are hereby incorporated as if individually set forth. Although the invention has been described by way of example and reference to possible embodiments thereof, it is to be appreciated that improvements and/or modifications may be made thereto without departing from the scope of the invention.

Aspects of the present invention have been described by way of example only and it should be appreciated that modifications and additions may be made thereto without departing from the scope thereof.

1. A method for extracting seeds from kiwifruit including the following steps:
Sterilizing the external surface of the fruit;
Separating the skin from the pulp and seed without damaging the seed coat;
Separating the seed from the pulp by soft mechanical screening;
Drying the isolated seed using low temperature methods.

2. A method of seed extraction as claimed in claim 1, wherein the seeds are extracted from Actinidia deliciosa or Actinidia cheninensis.

3. A method of seed extraction as claimed in claim 1, wherein the kiwifruit is sterilized by the use of a thermal process, followed by a chlorine wash and/or an ozone impregnated water wash and a fresh water rinse.

4. A method of seed extraction as claimed in claim 1, wherein a water laser is used to cut the fruit prior to the separation of the skin from the pulp.

5. A method of seed extraction as claimed in claim 1, wherein the seed and fruit pulp is separated from the skin by way of a twin converging belt press.

6. A method of seed extraction as claimed in claim 1, wherein the method comprises a further step of cleaning the seed after the seed is separated from the pulp by soaking the seed for up to 12 hours in a pectinase solution and then rinsing it with water on a screen.

7. A method of seed extraction as claimed in claim 1, wherein the seed is dried by freeze drying.

8. A method for extracting oil from kiwifruit seeds including the following steps:
Extracting seed from kiwifruit using the method as claimed in claim 1;
Crushing the seed immediately prior to oil extraction;
Using supercritical carbon dioxide extraction to extract the oil from the seed; and
Adding a natural antioxidant to the oil during or after extraction.

9. A method of oil extraction as claimed in claim 8, wherein natural rosemary oil is added to the kiwifruit oil during the supercritical carbon dioxide extraction process.

10. A method of oil extraction as claimed in claim 8, wherein the extracted kiwifruit seed oil is encapsulated into capsules.

11. A method of oil extraction as claimed in claim 8, wherein the extracted kiwifruit seed oil is dried onto an inert carrier.

12. Kiwifruit seed isolated according to the method as claimed in claim 1.

13. Kiwifruit seed oil manufactured according to the method as claimed in claim 8.
